(19) United States
(12) Patent Application Publication
(10) Pub. No.: US 2008/0045605 A1
(43) Pub. Date: Feb. 21, 2008

(54) Image Quality in the Eye

(76) Inventor: John Barbur, Berkhamsted (GB)

Correspondence Address: NIXON & VANDERHYE, PC, 901 NORTH GLEBE ROAD, 11TH FLOOR, ARLINGTON, VA, 22203, US

(21) Appl. No.: 11/629,997

(22) PCT Filed: Jun. 8, 2005
(86) PCT No.: PCT/EP05/06168

§ 371 Date: Feb. 1, 2007

(30) Foreign Application Priority Data
Jun. 29, 2004 (EP) 04015189.6

(51) Int. Cl. A61K 31/047; A61P 27/02

(52) U.S. Cl. 514/729

(57) ABSTRACT

Pharmaceutical compositions or dietary supplements are provided containing lutein, zeaxanthin, or mixtures thereof in amounts effective to improve image quality in the human eye. Methods are also provided for reducing higher order wavefront aberrations in the human eye to improve image quality by administering to a person in need of such treatment an effective amount of lutein, zeaxanthin or mixtures thereof.

The present invention relates to the improvement of image quality in the eye. The term “image quality” as used herein refers to the image formed on the retina. This image can be deteriorated by higher-order aberrations such as spherical aberrations, coma and irregular astigmatism.

While lower-order aberrations limit how much we can see, higher-order aberrations determine how well we can see. Higher-order aberrations are aberrations of the optics of the eye above and beyond nearsightedness, farsightedness and astigmatism. Higher-order aberrations cannot be corrected easily with glasses or contact lenses. Higher-order wavefront aberrations describe small deviations of the wavefront surface from the ideal surface and the reproduction of these deviations require a large number of components. Two common and potentially disruptive higher order aberrations are “spherical aberration” and “coma” see Liang J, Williams D R; Aberrations and retinal image quality of the normal human eye; J. Opt. Soc. Am. A. 1997; 14 (11):2873-83). For the visual benefits of correcting higher-order aberrations of the eye see also Williams et al., Visual Benefit of Correcting Higher Order Aberrations of the Eye, J. Refract. Surg. 2001, 16 (September/October 2000) S554-S559.

In accordance with the present invention it has been found that the administration of lutein and/or zeaxanthin leads to a reduction of higher-order wavefront aberrations in the eye. Thus, in one aspect, the invention relates to the use of lutein or zeaxanthin, or mixtures thereof, in the manufacture of a composition for reducing higher-order wavefront aberrations in the human eye. In another aspect, the present invention relates to a method of reducing higher-order wavefront aberrations in the human eye which comprises administering to a person in need of such treatment an effective amount of lutein or zeaxanthin, or mixtures thereof.

The term “higher order wavefront aberrations in the eye” as used herein denotes aberrations of the optical system of the eye that are distinct from myopia, hyperopia and axial astigmatism but nevertheless influence the quality of the image formed on the retina. They are measured by commercially available apparatuses, and are expressed as the wavefront function, which completely describes the aggregate effects of all refractive parts of the eye on light passing through every location of the pupil.

The wavefront aberration function is a way of quantifying the imperfections of the optics of the eye that cause degradation of retinal image quality. The “ideal” wavefront is generated when a “point” on the retina is imaged through the optics of the eye as a plane and flat surface just outside the eye. This means that all the rays emerging from the eye form a parallel bundle and are perpendicular to this plane. The “real” wavefront surface is not a perfect two-dimensional plane and reflects the aberrations present in the optical system. It is usually represented mathematically by a series of terms known as Zernike polynomials. A knowledge of the coefficients of the Zernike polynomials provides a complete description of the wavefront surface. In order to obtain one single number that describes the quality of the imaging system (in this case the human eye) the root mean square (rms) deviation is calculated by computing the root of the mean of the squares of the actual deviations from the ideal plane. The rms value is equivalent to a standard deviation, but the two functions compared are 2D surfaces (i.e., the ideal wavefront and the actual wavefront). The wavefront aberrations are measured with a wavefront analyzer (see, e.g., J. Refract. Surg. 2001; 17(5), S608-S612). The rms wavefront aberration increases significantly with the diameter of the pupil and can show large inter-subject variability. A small wavefront aberration value means better image quality on the retina and this translates to improved visual performance, (e.g., ability to see smaller or very faint objects, sharper and crisp images).

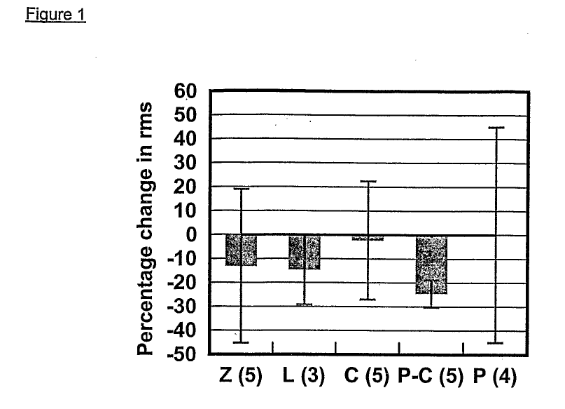

The reduction of higher-order wavefront aberrations in the human eye on administration of lutein and/or zeaxanthin can be seen from FIG. 1.

In FIG. 1 the number in parentheses is the number of subjects treated for 6 months with 20 mg/day zeaxanthin (Z), 20 mg/day lutein (L), 10 mg lutein+10 mg zeaxanthin per day (C, P—C), or placebo (P).

The term “composition” as used herein denotes any composition that is suitable for administration to the human body, such as pharmaceutical preparations or dietary supplements in dosage unit form, or a food, or a beverage.

A pharmaceutical preparation or dietary supplement in accordance with the present invention may be in any form that is conventional for oral administration, e.g. in solid form such as tablets including effervescent tablets, or soft or hard shell capsules, or in liquid form, such as solutions or suspensions, preferably oily suspension. Besides the active ingredients these preparations may contain conventional carrier material, additives and adjuvants, which include water, gelatin, vegetable gums, sugars, vegetable oils, polyalkylene glycols, flavoring agents, preservatives, stabilizers, emulsifying agents, buffers and the like. The medicaments may be in the form of controlled (delayed) release formulations. For the purpose of the invention the colorants as well as optional ingredients as defined earlier hereinabove may be incorporated in food or beverages, such as bakery items, e.g., cake and cookies, lemonades and fruit juices. The compositions according to the present invention may further contain physiologically active ingredients conventionally used to promote health, especially eye health, such as vitamins e.g. vitamin A, C and E, and minerals, such as selenium or zinc.

A suitable daily dosage of lutein and/or zeaxanthin for the purposes of the present invention may be within the range from 0.001 mg per kg body weight to about 20 mg per kg body weight. More preferred is a daily dosage of about 0.01 to about 10 mg per kg body weight, and especially preferred is about 0.1 to 1.0 mg per kg body weight per day.

In a preferred aspect, the invention relates to the use of a combination of lutein and zeaxanthin. In such combination these compounds are preferably used in a ratio of 0.1-1.0:1.0-0.1 parts by weight.

In solid dosage unit preparations, lutein and/or zeaxanthin are suitably present in an amount from about 0.1 mg to about 500 mg, preferably from about 1 mg to about 100 mg per dosage unit. In liquid formulations, the aforesaid ingredients are suitably present in an amount of from about 0.1 to about 5 percent by weight based upon the total weight of the composition.

Preferred solid dosage unit preparations comprise, per dosage unit, about 6 mg to about 12 mg of lutein and/or zeaxanthin.

The invention is illustrated further by the Examples given below:

### EXAMPLE 1

A soft gelatin capsule may be prepared comprising the following ingredients:

| Ingredient | Amount per Capsule |
|---|---|
| Lutein | 10 mg |
| Lecithin | 50 mg |
| Soy bean oil | 200 mg |

### EXAMPLE 2

A soft gelatin capsule may be prepared comprising the following ingredients:

| Ingredient | Amount per Capsule |
|---|---|
| Lutein | 10 mg |
| Zeaxanthin | 10 mg |
| Lecithin | 50 mg |
| Soy bean oil | 200 mg |

### EXAMPLE 3

A soft gelatin capsule may be prepared comprising the following ingredients:

| Ingredient | Amount per Capsule |
|---|---|
| Lutein | 6 mg |
| Zeaxanthin | 6 mg |
| Vitamin E (α-d,l-tocopherol) | 200 mg |
| Vitamin C | 500 mg |
| Lecithin | 50 mg |
| Soy bean oil | 200 mg |

### EXAMPLE 4

A soft gelatin capsule may be prepared comprising the following ingredients:

| Ingredient | Amount per Capsule |
|---|---|
| Lutein | 12 mg |
| Vitamin E (α-d,l-tocopherol) | 200 mg |
| Vitamin C | 500 mg |
| Lecithin | 50 mg |
| Soy bean oil | 200 mg |

### EXAMPLE 5

A soft gelatin capsule may be prepared comprising the following ingredients:

| Ingredient | Amount per Capsule |
|---|---|
| Zeaxanthin | 12 mg |
| Vitamin E (α-d,l-tocopherol) | 200 mg |
| Vitamin C | 500 mg |
| Lecithin | 50 mg |
| Soy bean oil | 200 mg |

### EXAMPLE 6

A soft gelatin capsule may be prepared comprising the following ingredients:

| Ingredient | Amount per Capsule | |
|---|---|---|
| Lutein | 6 | mg |
| Zeaxanthin | 6 | mg |
| β-Carotene | 6 | mg |
| Vitamin E (α-d,l-tocopherol) | 200 | mg |
| Vitamin C | 500 | mg |
| Zinc (as orotate) | 7.5 | mg |
| Lecithin | 50 | mg |
| Soy bean oil | 200 | |

### EXAMPLE 7

A soft gelatin capsule may be prepared comprising the following ingredients:

| Ingredient | Amount per Capsule | |
|---|---|---|
| Lutein | 6 | mg |
| Zeaxanthin | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

### EXAMPLE 8

A soft gelatin capsule may be prepared comprising the following ingredients:

| Ingredient | Amount per Capsule | |
|---|---|---|
| Lutein | 6 | mg |
| Zeaxanthin | 6 | mg |
| β-Carotene | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

1.-6. (canceled)

7. A method of reducing higher order wavefront aberrations in the human eye to improve image quality which comprises administering to a person in need of such treatment an effective amount of lutein or zeaxanthin, or mixtures thereof.

8. The method according to claim 7 wherein lutein is administered.

9. The method according to claim 7 wherein zeaxanthin is administered.

10. A method according to claim 7 wherein the daily dosage of lutein or zeaxanthin, or mixtures thereof is within the range of from 0.001 mg per kg body weight to about 20 mg per kg body weight, preferably from about 0.01 to about 10 mg per kg body weight, and most preferably from about 0.1 to 1.0 mg per kg body weight per day.

11. A pharmaceutical composition or dietary supplement in dosage unit form comprising an amount of lutein, zeaxanthin or mixtures thereof effective to improve image quality in a human eye.

12. A pharmaceutical composition or dietary supplement as in claim 11, wherein the amount of lutein, zeaxanthin or mixtures thereof is effective to reduce higher order wavefront aberrations in the human eye.

13. A pharmaceutical composition or dietary supplement as in claim 11, wherein the dosage unit form is for oral administration.

14. A pharmaceutical composition or dietary supplement as in claim 13, wherein the dosage unit form comprises an amount of about 0.1 mg to about 500 mg of lutein, zeaxanthin or mixtures thereof.

15. A pharmaceutical composition or dietary supplement as in claim 13, wherein the dosage unit form is a food or beverage.
